SEQUENCE LISTING (38 sequences)

SEQ ID NO: 1 - DNA, 19 bp, Artificial Sequence
  Synthetic oligonucleotide
gaatggacttctcatctga

SEQ ID NO: 2 - DNA, 20 bp, Artificial Sequence
  Synthetic oligonucleotide
gaactaagctcatcttgtct

SEQ ID NO: 3 - DNA, 20 bp, Artificial Sequence
  Synthetic oligonucleotide
gagactggctattgggggag

SEQ ID NO: 4 - DNA, 19 bp, Artificial Sequence
  Synthetic oligonucleotide
gaccgaaatgcttccaggg

SEQ ID NO: 5 - DNA, 23 bp, Artificial Sequence
  Synthetic oligonucleotide
cctacataaagcacctagatggc

SEQ ID NO: 6 - DNA, 22 bp, Artificial Sequence
  Synthetic oligonucleotide
atgtgatagtagatccaggcgt

SEQ ID NO: 7 - DNA, 21 bp, Artificial Sequence
  Synthetic oligonucleotide
caggacatccgccttaactgt

SEQ ID NO: 8 - DNA, 23 bp, Artificial Sequence
  Synthetic oligonucleotide
aggaagtaagtacccattagcca

SEQ ID NO: 9 - DNA, 23 bp, Artificial Sequence
  Synthetic oligonucleotide
ctgagatgtcacttcacatggaa

SEQ ID NO: 10 - DNA, 20 bp, Artificial Sequence
  Synthetic oligonucleotide
gtgcatccccaatgggttct

SEQ ID NO: 11 - DNA, 23 bp, Artificial Sequence
  Synthetic oligonucleotide
agtacaacgagtaaggagtcact

SEQ ID NO: 12 - DNA, 21 bp, Artificial Sequence
  Synthetic oligonucleotide
aggccagtatgttgcacatgg

SEQ ID NO: 13 - DNA, 22 bp, Artificial Sequence
  Synthetic oligonucleotide
aagagtcccctgcgattatttg

SEQ ID NO: 14 - DNA, 21 bp, Artificial Sequence
  Synthetic oligonucleotide
tctggatggcgtttgaattgg

SEQ ID NO: 15 - DNA, 21 bp, Artificial Sequence
  Synthetic oligonucleotide
cagaccaccgagatggactac

SEQ ID NO: 16 - DNA, 22 bp, Artificial Sequence
  Synthetic oligonucleotide
caaagactctcttgtttgctgc

SEQ ID NO: 17 - DNA, 21 bp, Artificial Sequence
  Synthetic oligonucleotide
ctcatcagcccgtggtctaaa

SEQ ID NO: 18 - DNA, 23 bp, Artificial Sequence
  Synthetic oligonucleotide
caccgccttaccaatatcttcaa

SEQ ID NO: 19 - DNA, 21 bp, Artificial Sequence
  Synthetic oligonucleotide
aagtcaggcgtctacaagtgg

SEQ ID NO: 20 - DNA, 21 bp, Artificial Sequence
  Synthetic oligonucleotide
cctccctttcttgagggtctt

SEQ ID NO: 21 - DNA, 23 bp, Artificial Sequence
  Synthetic oligonucleotide
agatcaacacctgtggtaacacc

SEQ ID NO: 22 - DNA, 20 bp, Artificial Sequence
  Synthetic oligonucleotide
ctctagggcctccacgaaca

SEQ ID NO: 23 - DNA, 21 bp, Artificial Sequence
  Synthetic oligonucleotide
gcttgttgggaaccctgtttg

SEQ ID NO: 24 - DNA, 23 bp, Artificial Sequence
  Synthetic oligonucleotide
ggatggcatttgttgatgtggag

SEQ ID NO: 25 - DNA, 25 bp, Artificial Sequence
  Synthetic oligonucleotide
ctcatggactgattatggacaggac

SEQ ID NO: 26 - DNA, 25 bp, Artificial Sequence
  Synthetic oligonucleotide
gcaggtcagcaaagaacttatagcc

SEQ ID NO: 27 - DNA, 24 bp, Artificial Sequence
  Synthetic oligonucleotide
gattggtggaagtttagctagcat

SEQ ID NO: 28 - DNA, 25 bp, Artificial Sequence
  Synthetic oligonucleotide
tagcattctcataagccaattgcat

SEQ ID NO: 29 - DNA, 20 bp, Artificial Sequence
  Synthetic oligonucleotide
tttgggacacaatgaaagca

SEQ ID NO: 30 - DNA, 20 bp, Artificial Sequence
  Synthetic oligonucleotide
ctgccgtctactcctcttgg

SEQ ID NO: 31 - DNA, 24 bp, Artificial Sequence
  Synthetic oligonucleotide
cttgcccttaaaggtctaaaagca

SEQ ID NO: 32 - DNA, 29 bp, Artificial Sequence
  Synthetic oligonucleotide
gcggtataaggtacaattaaaagatatgg

SEQ ID NO: 33 - DNA, 22 bp, Artificial Sequence
  Synthetic oligonucleotide
tttctcaaggcccaccaatagt

SEQ ID NO: 34 - DNA, 25 bp, Artificial Sequence
  Synthetic oligonucleotide
gacaccttttttaactatgcgagct

SEQ ID NO: 35 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 36 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 37 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 38 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

(19) United States
(12) Patent Application Publication
(10) Pub. No.: US 2024/0277810 A1
(43) Pub. Date: Aug. 22, 2024

(54) INTERFERONS AND NUCLEAR EXPORT INHIBITORS FOR USE IN METHODS OF TREATING CANCER

(71) Applicant: St. Jude Children's Research Hospital, Inc., Memphis, TN (US)

(72) Inventors: Thirumala-Devi KANNEGANTI, Memphis, TN (US); Rajendra KARKI, Memphis, TN (US)

(21) Appl. No.: 18/565,700

(22) PCT Filed: May 25, 2022
(86) PCT No.: PCT/US2022/030857

§ 371 Date: Nov. 30, 2023

Related U.S. Application Data
(60) Provisional application No. 63/196,986, filed on Jun. 4, 2021.

(51) Int. Cl. A61K 38/21; A61K 31/366; A61K 31/497; A61P 35/00

(52) U.S. Cl. CPC A61K 38/21; A61K 31/366; A61K 31/497; A61P 35/00

(57) ABSTRACT

Methods for inducing inflammatory cell death and treating cancer are provided, which include the targeting of a component of the ZBP1-ADAR1 PANoptosis pathway and the use of a combination of one or more interferons or one or more agents that upregulate interferon production and one or more nuclear export inhibitors.

### INTRODUCTION

This application claims benefit of priority to U.S. Provisional Patent Application Ser. No. 63/196,986, filed Jun. 4, 2021, the content of which is incorporated herein by reference in its entirety.

This invention was made with government support under Grant Numbers AI101935, AI124346, AR056296, AI160179 and CA253095 awarded by the National Institutes of Health. The government has certain rights in the invention.

### BACKGROUND

Nucleic acids, particularly dsRNA from pathogens, are sensed by cytosolic RNA sensors to produce type I interferons (IFNs), which provide host defense against invading pathogens. However, some endogenous RNAs, such as short interspersed nuclear elements, contain RNA duplexes that can also be recognized, leading to sustained and profound IFN production. To avoid this pathological overactivation of the immune system, mammalian cells contain an evolutionarily conserved post-transcriptional RNA base modification catalyzed by the protein adenosine deaminase acting on RNA 1 (ADAR1) that prevents the sensing of endogenous dsRNA. ADAR1 is critical for development and survival. Mutations in ADAR1 and the subsequent sustained type I IFN response have been associated with several autoimmune and autoinflammatory disorders such as Aicardi-Goutieres syndromes (AGS), systemic lupus erythematosus, and bilateral striatal necrosis. In addition, although the underlying mechanisms remain unclear, recent studies have suggested an association between ADAR1 and tumorigenesis.

Among the major isoforms of ADAR1, ADAR1-p150 is the IFN-inducible form and shuttles between the nucleus and cytoplasm. Moreover, ADAR1-p150 contains a Z-alpha (Zα) domain. Besides ADAR1, the only other mammalian protein that contains Zα domains is Z-DNA binding protein 1 (ZBP1), which is known to recognize viral and endogenous Z-RNAs. The Zα domain of ZBP1 recognizes Z-RNAs to activate the NLRP3 inflammasome and inflammatory cell death, characterized by pyroptosis, apoptosis, and necroptosis (PANoptosis) in myeloid cells. Although both ADAR1-p150 and ZBP1 contain Zα domains and are IFN-inducible, whether ADAR1 functions similarly to ZBP1 is unclear.

In addition to its Zα domain, ADAR1-p150 also contains a nuclear export signal (NES) within the Zα domain. Chromosomal maintenance 1 (CRM1), also known as exportin 1 (XPO1), mediates the export of ADAR1-p150 to the cytoplasm using its NES. Mutation of the NES or treatment with nuclear export inhibitors (NEIs) specific to XPO1 cause nuclear accumulation of ADAR1-p150 and cell death (Poulsen, et al. (2001) Mol. Cell Biol. 21:7862-71). Whether this is similar to the cell death induced by ZBP1 is unknown. Similar to ADAR1, XPO1 overexpression correlates with poor prognosis in various cancers (Taylor, et al. (2019) Cancer Discov. 9:1452-1467). Treatments targeting XPO1, such as selective NEIs, are beneficial in cancer treatment (Azizian & Li (2020) J. Hematol. Oncol. 13:61). NEIs such as leptomycin B (LMB) or selinexor (KPT-330) have anti-tumor efficacy in several preclinical models of solid tumor and hematological malignancies (Gravina, et al. (2014) J. Hematol. Oncol. 7:8). Moreover, KPT-330 has received FDA approval for use in patients with relapsed/refractory multiple myeloma (Chari, et al. (2019) N. Engl. J. Med. 381:727-738; Theodoropoulos, et al. (2020) Target Oncol. 15:697-708) or diffuse large B-cell lymphoma (Kalakonda, et al. (2020) Lancet Haematol. 7:e511-522).

### SUMMARY OF THE INVENTION

This invention provides a composition and method for treating cancer, which include an effective amount of (i) one or more interferons (e.g., IFN-α, IFN-β, and/or IFN-γ), or (ii) or one or more compounds that upregulate interferon production (e.g., amidobenzimidazoles, flavone acetic acid analogues or cyclic dinucleotides); and one or more nuclear export inhibitors (e.g., a leptomycin, a ratjadone, an anguinomycin, callystatin, valtrate, oridonin, acetoxychavicol acetate, curcumin, gonionthalamin, piperlongumine, plumbagin, CBS-9106, KPT-185, KPT-249, KPT-251, KPT-276, KPT-301, KPT-330, KPT-335 and/or KPT-8602). In some aspects, the cancer is a head and neck cancer, liver cancer, intestinal cancer, ovarian cancer, uterine cancer, testicular cancer, bladder cancer, gastric cancer, colorectal cancer, pancreatic cancer, thyroid cancer, kidney cancer, prostate cancer, melanoma, lung cancer, breast cancer, sarcoma, cancer of the central nervous system, lymphoma, leukemias or myeloma.

### BRIEF DESCRIPTION OF THE DRAWINGS

FIG. 1 shows that interferons potentiate the cell death induced by nuclear export inhibitors. Wild-type BMDMs were treated with KPT-330 or leptomycin B for 24 hours in the presence or absence of IFN-β or IFN-γ and cell death was quantified by staining with propidium iodide (PI). Data are representative of at least three independent experiments. ****P<0.0001. Analysis was performed using the two-way ANOVA. Data are shown as mean±SEM.

FIG. 2 shows that ZBP1 triggers inflammatory cell death, PANoptosis, in response to the combination of IFNs and nuclear export inhibitors. Wild-type, Zbp1^{-/-} and Zbp1^{ΔZα2} BMDMs were treated with KPT-330 or leptomycin B for 24 hours in the presence of IFN-β and cell death was quantified by staining with PI. Data are representative of at least three independent experiments. ****P<0.0001. Analysis was performed using the two-way ANOVA. Data are shown as mean±SEM.

FIG. 3A-3C show that treatment with IFN-α (FIG. 3A), IFN-β (FIG. 3B), or IFN-γ (FIG. 3C) in combination with KPT-330 significantly regresses tumors in vivo. Melanoma was induced with the engraftment of B16-F10 melanoma cells by subcutaneously injecting 1×10⁶ cells. For treatment with IFN-γ, each mouse was injected interperitoneally with 10 μg of IFN-γ. For treatment with IFN-α or IFN-β, each mouse was injected interperitoneally with 1 μg of IFN-α or IFN-β. For treatment with KPT-330 (Selleckchem), each mouse was orally administered 300 μl of KPT-330 (15 mg/kg). The treatment was given on days 8, 10 and 13 after tumor implantation. **P<0.01, ****P<0.0001. Data are shown as mean±SEM.

### DETAILED DESCRIPTION OF THE INVENTION

This invention provides for the use of interferons (IFNs) in combination with nuclear export inhibitors (NEIs) to induce robust cell death. In particular, combining IFNs such as IFN-α, IFN-β, or IFN-γ with NEIs such as KPT-330 or leptomycin B induces robust inflammatory cell death (PANoptosis), while IFNs or NEIs alone induce a low level of cell death. Notably, the cell death induced by the combination of IFNs and NEIs is dependent on ZBP1 and RIPK3, and the interaction of ZBP1 with the Zα domain of ADAR1. Whereas loss of ADAR1 or sequestration of ADAR1 to the nucleus increases the interaction between ZBP1 and RIPK3 thereby leading to cell death, loss of RIPK3 increases the interaction between ADAR1 and ZBP1 thereby blocking cell death. Expression of ZBP1 and ADAR1 are induced upon IFN treatment, and ZBP1 is sequestered by ADAR1 in IFN-stimulated cells, preventing cell death. Mice lacking ADAR1 expression in myeloid cells are resistant to the development of colorectal cancer and melanoma. The success of IFN cancer therapies has been hindered by tumor resistance, and the present analyses indicate that this may be due to the induction of ADAR1-p150 and ZBP1 expression simultaneously by IFN. In this respect, sequestering ADAR1-p150 within the nucleus by the use of NEIs potentiates the efficacy of IFN therapy for cancer treatment, in particular tumors exhibiting resistance to IFN treatment alone. Accordingly, the present invention provides methods for treating cancer and inducing inflammatory cell death by targeting components of the ZBP1-ADAR1 PANoptosis pathway described herein. In particular, the invention provides for the administration of an IFN and/or agent that induces IFN production in combination with a nuclear export inhibitor, where the combined administration provides a synergistic effect in inducing inflammatory cell death, elimination of cancer cells, and treatment of cancer.

For the purposes of this invention, the term “interferon” refers to a full-length interferon or to an interferon fragment (truncated interferon) or interferon mutant, that substantially retains the biological activity of the full length wild-type interferon (e.g., retains at least 50%, or preferably at least 60%, or preferably at least 70%, or preferably at least 80%, preferably at least 90%, more preferably at least 95%, 98%, or 99% of the full-length interferon in its isolated form.

There are seven classes of type I IFNs with IFN-α and IFN-β being the most abundant. Both IFN-α and IFN-β bind to the same receptor composed of two transmembrane proteins, IFNAR 1 and 2, but IFN-β binds with much higher affinity than IFN-α (Lamken, et al. (2004) J. Mol. Biol. 341:303-318). Interferons of use in this invention include type I interferons (e.g., IFN-α and IFN-β) as well as type II interferons (e.g., IFN-γ). The interferon can be from essentially any mammalian species. Ideally, the interferon is from a human, equine, bovine, rodent, porcine, lagomorph, feline, canine, murine, caprine, ovine, a non-human primate, and the like. The amino acid sequences of human interferons are known in the art and available under GENBANK Accession Nos. NP 076918.1 (IFN-α; SEQ ID NO:35), NP_000596.1 (IFN-α2; SEQ ID NO:36), NP 002167.1 (IFN-β; SEQ ID NO:37), and NP_000610.2 (IFN-γ; SEQ ID NO:38).

In some aspects, the interferon is mutated, wherein said mutation includes one or more amino acid substitutions, insertions, and/or deletions that improve activity, receptor binding, expression, solubility, stability, glycation, half-life and/or administration, yet said mutant retains the desired activity. A mutant IFN-α may include, e.g., mutated IFN-α2 or IFN-α2^{YNS} having the mutations H57Y, E58N, and Q61S (YNS) (see, e.g., Kalie et al. (2007) J. Biol. Chem. 282:11602-11611), which have a higher affinity for IFNAR. A mutated IFN-β having a serine substituted for the naturally occurring cysteine at amino acid 17 has also been demonstrated to show efficacy (see, e.g., Hawkins, et al. (1985) Cancer Res. 45:5914-5920). In addition, R27T and V101F mutations have been shown to improve human IFN-β solubility and half-life without interfering with binding to the receptor (Balkhi & Hajati (2019) Adv. Pharm. Bull. 9(4):640-8).

In other aspects, truncated interferons are used in method of this invention. Human INF-α, for example, with deletions of the first 15 amino-terminal amino acid residues and/or the last 10-13 carboxyl-terminal amino acid residues, have been shown to exhibit virtually the same activity as the parent molecules (see, e.g., Ackerman (1984) Proc. Natl. Acad. Sci. USA 81:1045-1047). Accordingly, the use of IFN-ca molecules having 1, 2, 3, up to 13 carboxyl terminal amino acid residues deleted and/or 1, 2, 3, up to 15 amino terminal amino acid residues deleted are contemplated. It has also been demonstrated that activity resides in the fragment, HuIFN-α (1-110). Accordingly, carboxyl truncated IFNs with truncations after residue 110 and/or with 1, 2, 3, up to 15 amino terminal amino acid residues deleted are contemplated.

Certain C-terminally truncated IFN-βs have been shown to have increased activity (see, e.g., U.S. Pat. No. 7,915,483). Accordingly, in certain aspects the interferon used in the methods described herein includes the C-terminally truncated IFN-β referred to as IFN-Δ1, IFN-Δ2, IFN-Δ3, IFN-Δ4, IFN-Δ5, IFN-Δ6, IFN-Δ7, IFN-Δ8, IFN-Δ9, or IFN-Δ10 in U.S. Pat. No. 7,915,483, incorporated herein by reference in its entirety.

In certain aspects, a chemically modified interferon can be used. For example, in certain aspects, the interferon is chemically modified to increase serum half-life. For example, (2-sulfo-9-fluorenylmethoxycarbonyl)₇-interferon-α2 undergoes time-dependent spontaneous hydrolysis, generating active interferon (see, e.g., Shechter, et al. (2001) Proc. Natl. Acad. Sci. USA 98(3):1212-1217). Other modifications include for example N-terminal modifications including, but not limited to, the addition of polyethylene glycol (PEG), protecting groups, and the like. See U.S. Pat. No. 5,824,784, incorporated herein in its entirety, for N-terminally chemically modified interferon.

Suitable interferons of use in this invention are known in the art and commercially available under the tradenames AVONEX® (IFN-β1a), REBIF® (IFN-β1a), PLEGRIDY® (IFN-β1a), BETASERON® (IFN-β1b), REBIF® REBIDOSE® (IFN-β1a), INFERGEN™ (IFN alfacon-1), EXTAVIA® (IFN-β1b), ALFERON® N (IFN-αn3), PEGASYS® (IFN-α2a), INTRON® A (IFN-α2b), ROFERON™-A (IFN-α2a), and ACTIMMUNE® (IFN-γ1b).

In some aspects, the invention further provides for the use of one or more agents that upregulate interferon production in combination with one or more nuclear export inhibitors. Agents that upregulate interferon production refer to compounds or molecules that induce the expression (transcription and/or translation of interferon). In certain aspects, agents that upregulate interferon production are small organic compounds. For example, endogenous interferon production can be upregulated in cells upon treatment with stimulator of interferon gene (STING) agonists. In particular, the STING agonist linked amidobenzimidazoles (diABZIs) upregulate the production of interferon in cells (see, e.g., Li, et al. (2021) Sci. Immunol. 6(59):eabi9007). Other exemplary agents that upregulate interferon production include, but are not limited to, flavone acetic acid analogues such as 5,6-dimethylxanthenone-4-acetic acid; and cyclic dinucleotides such as cyclic dimeric guanosine monophosphate (c-di-GMP), cyclic dimeric adenosine monophosphate (c-di-AMP), and/or cyclic GMP-AMP (cGAMP).

Nuclear export inhibitors (NEIs) are compounds that block Chromosomal maintenance 1 (CRM1), also known as exportin 1 (XPO1), a protein involved in transport of biopolymers from the cell nucleus to the cytoplasm, or otherwise prevent proteins from translocating out of the nucleus. NEIs, also referred to herein as CRM1 inhibitors, have been shown to bind covalently to Cys528 of CRM1 by a Michael-type addition reaction and abrogate the interaction between CRM1 and its cargo protein. The first CRM1 inhibitor to be discovered was leptomycin B (LMB), which is naturally made by Streptomyces bacteria. LMB was initially used as an anti-fungal agent, and later as an anti-cancer agent (Hamamoto, et al. (1985) J. Antibiot. (Tokyo) 38:1573-80; Lu, et al. (2012) PLoS One 7:e32895). Additional natural NEIs have been described including leptomycin A, ratjadone A, ratjadone C, anguinomycin A, anguinomycin B, anguinomycin C, anguinomycin D, 15d-PGJ2, and callystatin, which are polyketides. Several plant NEIs were discovered from South/Southeast Asia herbs and food additives, and include, e.g., valtrate, oridonin, acetoxychavicol acetate, curcumin, gonionthalamin, piperlongumine and plumbagin.

The next generation of NEIs to be developed were collectively known as selective inhibitors of nuclear transport (SINE) compounds and include 1-((6-chloro-5-(trifluoromethyl)pyridin-2-yl)amino)-3-((3,3-dimethylbutoxy)methyl)-4-methyl-1H-pyrrole-2,5-dione (CBS-9106), KPT-185, KPT-249, KPT-251 ((Z)-2-(2-(3-(3,5-bis(trifluoromethyl)phenyl)-1H-1,2,4-triazol-1-yl)vinyl)-1,3,4-oxadiazole), KPT-276, KPT-301, KPT-330 (selinexor), KPT-335 (verdinexor) and KPT-8602 (eltanexor). See, e.g., U.S. Pat. No. 9,428,490, incorporated herein by reference in its entirety. Similar to their predecessors, these molecules form covalent bonds to Cys528 on CRM1. However, they improve upon the first-generation compounds by engaging in a slowly reversible covalent bonding, which improves upon the toxicity profile. KPT-330, the most widely used SINE, has been described for use in the treatment of lymphoma (i.e., Non-Hodgkin's lymphoma, diffuse large B-cell lymphoma), sarcomas, lung cancer, gliomas, breast cancer, leukemia (ALL, AML, MDS), multiple myeloma (MM), gastric cancer, pancreatic cancer, esophageal cancer, prostate cancer, melanoma, colorectal cancer, thymic cancer, and gynecologic cancers.

The structures of natural and synthetic NEIs are presented in Table 1.

TABLE 1

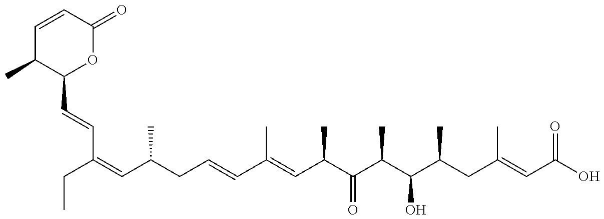 [SMILES: CCC(=C/[C@H](C)C/C=C/C(C)=C/[C@@H](C)C(=O)[C@@H](C)[C@H](O)[C@@H](C)C/C(C)=C/C(=O)O)/C=C/[C@@H]1OC(=O)C=C[C@@H]1C]

Leptomycin B

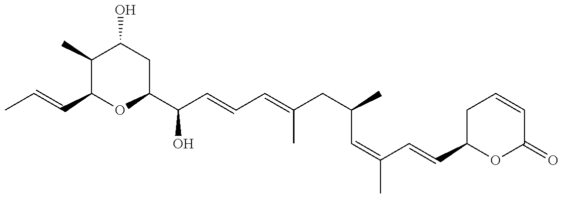 [SMILES: C/C=C/[C@@H]1O[C@H]([C@H](O)/C=C/C=C(\C)C[C@@H](C)/C=C(C)\C=C\[C@H]2CC=CC(=O)O2)C[C@@H](O)[C@@H]1C]

Ratjadone A

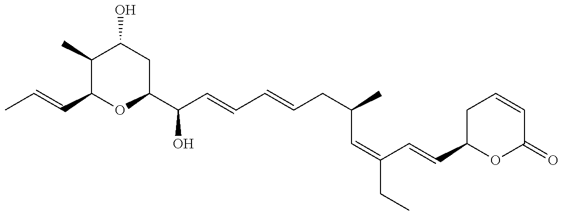 [SMILES: C/C=C/[C@@H]1O[C@H]([C@H](O)/C=C/C=C/C[C@@H](C)/C=C(\C=C\[C@H]2CC=CC(=O)O2)CC)C[C@@H](O)[C@@H]1C]

Ratjadone C

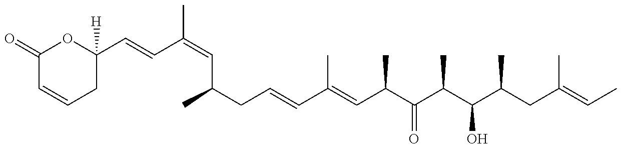 [SMILES: C/C=C(\C)C[C@H](C)[C@@H](O)[C@H](C)C(=O)[C@H](C)/C=C(C)/C=C/C[C@@H](C)/C=C(C)\C=C\[C@H]1CC=CC(=O)O1]

Anguinomycin C

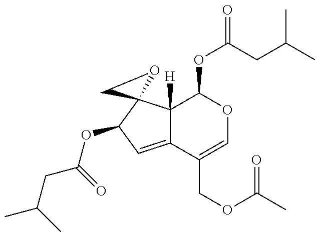 [SMILES: CC(=O)OCC1=CO[C@H](OC(=O)CC(C)C)[C@@H]2C1=C[C@@H](OC(=O)CC(C)C)[C@@]21CO1]

Valtrate

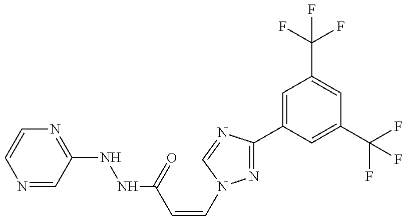 [SMILES: O=C(/C=C\n1cnc(-c2cc(C(F)(F)F)cc(C(F)(F)F)c2)n1)NNc1cnccn1]

KPT-330

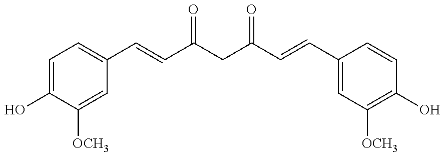 [SMILES: COc1cc(/C=C/C(=O)CC(=O)/C=C/c2ccc(O)c(OC)c2)ccc1O]

Curcumin

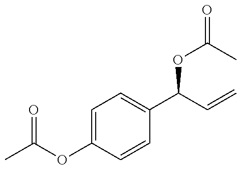 [SMILES: C=C[C@H](OC(C)=O)c1ccc(OC(C)=O)cc1]

Acetoxychavicol Acetate

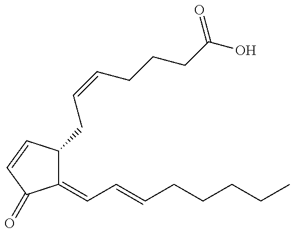 [SMILES: CCCCC/C=C/C=C1/C(=O)C=C[C@@H]1C/C=C\CCCC(=O)O]

15d-PGJ2

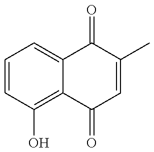

Plumbagin

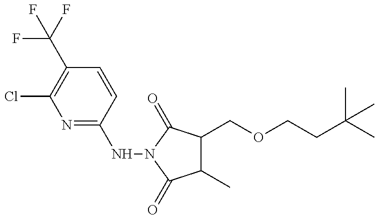 [SMILES: CC1C(=O)N(Nc2ccc(C(F)(F)F)c(Cl)n2)C(=O)C1COCCC(C)(C)C]

CBS9106

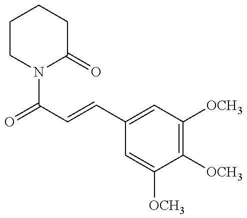 [SMILES: COc1cc(/C=C/C(=O)N2CCCCC2=O)cc(OC)c1OC]

Piperlongumine

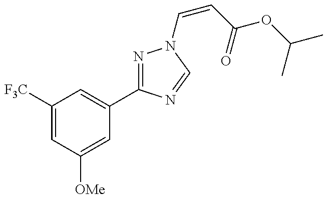 [SMILES: COc1cc(-c2ncn(/C=C\C(=O)OC(C)C)n2)cc(C(F)(F)F)c1]

KPT-185

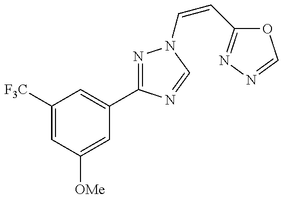 [SMILES: COc1cc(-c2ncn(/C=C\c3nnco3)n2)cc(C(F)(F)F)c1]

KPT-251

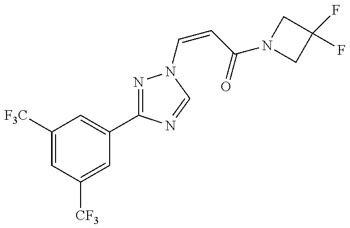 [SMILES: O=C(/C=C\n1cnc(-c2cc(C(F)(F)F)cc(C(F)(F)F)c2)n1)N1CC(F)(F)C1]

KPT-276

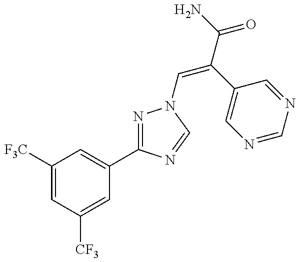 [SMILES: NC(=O)/C(=C/n1cnc(-c2cc(C(F)(F)F)cc(C(F)(F)F)c2)n1)c1cncnc1]

KPT-8602

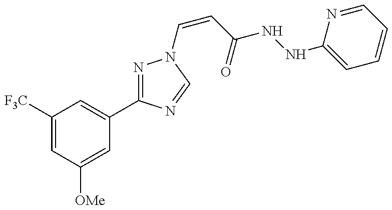 [SMILES: COc1cc(-c2ncn(/C=C\C(=O)NNc3ccccn3)n2)cc(C(F)(F)F)c1]

KPT-335

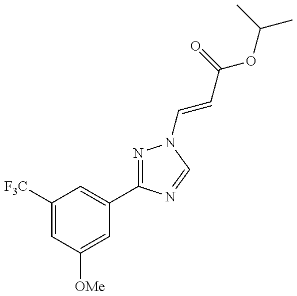 [SMILES: COc1cc(-c2ncn(/C=C/C(=O)OC(C)C)n2)cc(C(F)(F)F)c1]

KPT-301

When used in the methods of this invention, the interferon or agent that upregulates interferon production, and NET are administered to a patient in need of treatment in order to induce inflammatory cell death and/or prevent or treat cancer. The combination of agents may also be administered to cells in culture, e.g., in vitro or ex vivo, to diagnose and/or study cancer. In this respect, the activity of a combination of agents used in this invention may be assayed in vitro, in vivo, ex vivo or in a cell line.

As used herein, the term “treat” or “treatment” is defined as the application or administration of an effective amount of a combination of one or more interferons or one or more agents that upregulate interferon production with one or more NEIs, to a subject in need of treatment, e.g., a subject having cancer, a symptom of cancer, or a predisposition toward cancer in order to cure, heal, alleviate, relieve, alter, remedy, ameliorate, improve or affect the cancer, one or more symptoms of the cancer or the predisposition toward the cancer (e.g., to prevent at least one symptom of the cancer or to delay onset of at least one symptom of the cancer).

As used herein, an amount of a compound effective to treat cancer, or a “therapeutically effective amount” refers to an amount of the compound which is effective, upon single or multiple dose administration to a subject or a cell, in curing, alleviating, relieving, or improving one or more symptoms of cancer.

As used herein, an amount of a compound effective to prevent cancer, or a “prophylactically effective amount” of the compound refers to an amount effective, upon single- or multiple-dose administration to the subject, in preventing or delaying the onset or recurrence of cancer or one or more symptoms of cancer.

For the purposes of this invention, the term “subject” is intended to include human and non-human animals. Exemplary human subjects include a human patient having cancer. The term “non-human animals” of the invention includes all vertebrates, e.g., non-mammals (such as chickens, amphibians, reptiles) and mammals, such as non-human primates, domesticated and/or agriculturally useful animals, e.g., sheep, cow, pig, etc., and companion animals (dog, cat, horse, etc.).

In particular aspects, when the NEI is administered in combination with an interferon or agent that upregulates interferon, the invention provides for the use of a lower therapeutic dose of the nuclear export inhibitor and/or interferon/agent that upregulates interferon, thus significantly widening the therapeutic window for treatment. In one aspect, the therapeutic dose of nuclear export inhibitor and/or interferon/agent that upregulates interferon production is lowered by at least about 10%. In other aspects, the therapeutic dose of nuclear export inhibitor and/or interferon/agent that upregulates interferon production is lowered by about 10% to 20%, by about 20% to 50%, by about 50% to 200%, or by about 100% to 1000%.

In other aspects, the use of a nuclear export inhibitor in combination with an interferon/agent that upregulates interferon production provides for a synergistic effect. As used herein, the term “synergistic” refers to a combination of therapeutic agents that is more effective than the additive effect of two or more single agents. The determination of the synergistic interaction between one or more interferons/agents that upregulate interferon production and one or more NEIs can be based on the results obtained from the assays described herein. The results of these assays were analyzed using the Chou-Talalay combination method and CalcuSyn software dose-effect analysis to obtain a combination index (Chou & Talalay (1984) Adv. Enzyme Regul. 22:27-55). A combination index value of less than 0.8 indicates synergy, a value greater than 1.2 indicates antagonism and a value between 0.8 and 1.2 indicates a superposition effect. Combination therapies provide “synergistic effects” and prove to be “synergistic,” i.e., the effect achieved when the active ingredients are used together is greater than the sum of the effects caused by the use of the compounds alone. Synergistic effects may be obtained when the active ingredients are: (1) co-provisioned and simultaneously administered or delivered in a combined unit dose formulation; (2) alternately or in parallel as an independent formulation; or (3) take advantage of some other options. When delivered in alternation therapy, synergistic effects can be obtained, for example, by sequentially administering or delivering compounds by different injections in separate syringes. In general, during an alternation therapy, an effective amount of each active ingredient is administered sequentially (i.e., continuously), while in combination therapy, two or more active ingredients of an effective amount are administered simultaneously.

Accordingly, the methods of the present invention may be carried out in several basic ways. A subject may first be treated with a dose of one or more interferons/agents that upregulate interferon production and subsequently be treated with a dose of one or more nuclear export inhibitors. Alternatively, the subject may first be treated with a dose of one or more nuclear export inhibitors and subsequently be treated with a dose of one or more interferons/agents that upregulate interferon production. As a further alternative, the subject may be treated simultaneously with a dose of one or more nuclear export inhibitors and a dose of one or more interferons/agents that upregulate interferon production.

In some aspects, a subject is first treated with a dose of a nuclear export inhibitor. After waiting for a period of time sufficient to allow development of a substantially efficacious response of the nuclear export inhibitor, a formulation including a synergistic dose of an interferon/agent that upregulates interferon production together with a second sub-toxic dose of the nuclear export inhibitor is administered. In general, the appropriate period of time sufficient to allow development of a substantially efficacious response to the nuclear export inhibitor will depend upon the pharmacokinetics of the nuclear export inhibitor and will have been determined during clinical trials of therapy using the nuclear export inhibitor alone. In one aspect of the invention, the period of time sufficient to allow development of a substantially efficacious response to the nuclear export inhibitor is between about 1 hour and 96 hours. In another aspect of the invention, the period of time sufficient to allow development of a substantially efficacious response to the nuclear export inhibitor is between about 2 hours and 48 hours. In another aspect of the invention, the period of time sufficient to allow development of a substantially efficacious response to the nuclear export inhibitor is between about 4 hours and 24 hours. The appropriate dosing regimen may depend on the particular nuclear export inhibitors and/or interferons/agents that upregulate interferon production employed.

The combination of one or more interferons/agents that upregulate interferon production and one or more nuclear export inhibitors may allow for the use of a lower therapeutic dose of the nuclear export inhibitor and/or interferon/agent that upregulates interferon production for the treatment of cancer. That a lower dose of nuclear export inhibitor and/or interferon/agent that upregulates interferon production oftentimes lessens the side effects observed in a subject. The lessened side effects can be measured both in terms of incidence and severity. Severity measures are provided through a grading process delineated by the National Cancer Institute (common toxicity criteria NCI CTC, Version 2). For instance, the incidence of side effects is typically reduced 10%. Oftentimes, the incidence is reduced 20%, 30%, 40% or 50%. Furthermore, the incidence of grade 3 or 4 toxicities for more common side effects associated with a nuclear export inhibitor and/or interferon/agent that upregulates interferon production administration (e.g., anemia, anorexia, diarrhea, fatigue, nausea, and vomiting) is oftentimes reduced 10%, 20%, 30%, 40% or 50%.

Formulations used in the method of the invention may be in any suitable form, such as a solid, semisolid, or liquid form such as liquid solutions (e.g., injectable, and infusible solutions), dispersions or suspensions, tablets, pills, powders, liposomes, and suppositories. See, e.g., Gennaro (2000) “Remington: The Science and Practice of Pharmacy,” 20^{th} Edition, Lippincott, Williams & Wilkins; Ansel, et al. (1999) “Pharmaceutical Dosage Forms and Drug Delivery Systems,” 7^{th} Edition, Lippincott Williams & Wilkins Publishers; and Kibbe (2000) “Handbook of Pharmaceutical Excipients American Pharmaceutical Association,” 3^{rd} Edition. In general, the pharmaceutical preparation will contain one or more of the compounds of the present invention as an active ingredient in admixture with an organic or inorganic carrier or excipient suitable for external, enteral, or parenteral application. The active ingredient may be compounded, for example, with the usual non-toxic, pharmaceutically acceptable carriers for tablets, pellets, capsules, suppositories, pessaries, solutions, emulsions, suspensions, and any other form suitable for use. The carriers that can be used include water, glucose, lactose, gum acacia, gelatin, mannitol, starch paste, magnesium trisilicate, talc, corn starch, keratin, colloidal silica, potato starch, urea, and other carriers suitable for use in manufacturing preparations in solid, semi-solid, or liquefied form. In addition, auxiliary stabilizing, thickening, and coloring agents and perfumes may be used. In certain aspects, the nuclear export inhibitor and/or interferon/agent that upregulates interferon production are formulated with a preservative to prevent unwanted microbial growth. Where applicable, the compounds useful in the methods of the invention may be formulated as microcapsules and nanoparticles. General protocols are described, for example, by Microcapsules and Nanoparticles in Medicine and Pharmacy by Max Donbrow, ed., CRC Press (1992) and by in U.S. Pat. Nos. 5,510,118, 5,534,270, and 5,662,883. By increasing the ratio of surface area to volume, these formulations allow for the oral delivery of compounds that would not otherwise be amenable to oral delivery.

Another method involves encapsulating the compounds useful in the methods of the invention in liposomes. Methods for forming liposomes as drug delivery vehicles are well known in the art. Suitable protocols include those described in U.S. Pat. Nos. 5,683,715, 5,415,869, and 5,424,073. Of the various lipids that may be used, particularly preferred lipids for making encapsulated liposomes include phosphatidylcholine and polyethylene glycol-derivatized distearyl phosphatidyl-ethanolamine.

Yet another method involves formulating the compounds useful in the methods of the invention using polymers such as biopolymers or biocompatible (synthetic or naturally occurring) polymers. Biocompatible polymers can be categorized as biodegradable and non-biodegradable.

Biodegradable polymers degrade in vivo as a function of chemical composition, method of manufacture, and implant structure. Illustrative examples of synthetic polymers include polyanhydrides, polyhydroxy acids such as polylactic acid, polyglycolic acids and copolymers thereof, polyesters, polyamides, polyorthoesters and some polyphosphazenes. Illustrative examples of naturally occurring polymers include proteins and polysaccharides such as collagen, hyaluronic acid, albumin, and gelatin.

Another method involves conjugating the compounds useful in the methods of the invention to a polymer that enhances aqueous solubility, stability, delivery and or half-life. Examples of suitable polymers include polyethylene glycol, poly-(d-glutamic acid), poly-(1-glutamic acid), poly-(1-glutamic acid), poly-(d-aspartic acid), poly-(1-aspartic acid) and copolymers thereof. Polyglutamic acids having molecular weights between about 5,000 to about 100,000 are preferred, with molecular weights between about 20,000 and 80,000 being more preferred wand with molecular weights between about 30,000 and 60,000 being most preferred.

In another method, the compounds useful in the methods of the invention are conjugated to a monoclonal antibody. This method allows the targeting of the compounds to specific targets. General protocols for the design and use of conjugated antibodies are described in Monoclonal Antibody—Based Therapy of Cancer by Michael L. Grossbard, ED. (1998).

The preferred form to be administered depends, in part, on the intended mode of administration and therapeutic application. For example, compositions containing a composition intended for systemic or local delivery can be in the form of injectable or infusible solutions. Accordingly, the compositions can be formulated for administration by a parenteral mode (e.g., intravenous, subcutaneous, intraperitoneal, or intramuscular injection). “Parenteral administration,” “administered parenterally,” and other grammatically equivalent phrases, as used herein, refer to modes of administration other than enteral and topical administration, usually by injection, and include, without limitation, intravenous, intranasal, intraocular, pulmonary, intramuscular, intraarterial, intrathecal, intracapsular, intraorbital, intracardiac, intradermal, intrapulmonary, intraperitoneal, transtracheal, subcutaneous, subcuticular, intraarticular, subcapsular, subarachnoid, intraspinal, epidural, intracerebral, intracranial, intracarotid and intrasternal injection and infusion. In some aspects, the methods of the invention provide for intratumoral delivery (e.g., with an antibody-targeted approach or lipoparticle/nanoparticle).

The amount of active ingredients that may be combined with the carrier materials to produce a single dosage form will vary depending upon the subject treated and the particular mode of administration. For example, a formulation for intravenous use may include an amount of the interferon or NEI ranging from about 1 mg/mL to about 25 mg/mL, preferably from about 5 mg/mL, and more preferably about 10 mg/mL. Intravenous formulations are typically diluted between about 2-fold and about 30-fold with normal saline or 5% dextrose solution prior to use.

The methods of the present invention provide for the induction of inflammatory cell death and the treatment of cancer.

In one aspect, the methods of the present invention are used to treat cancers of the head and neck, which include, but are not limited to, tumors of the nasal cavity, paranasal sinuses, nasopharynx, oral cavity, oropharynx, larynx, hypopharynx, salivary glands, and paragangliomas. In another aspect, the method of the present invention includes the treatment of cancers of the liver and biliary tree, particularly hepatocellular carcinoma. In another aspect, the method of the present invention includes the treatment of intestinal cancers, particularly colorectal cancer. In another aspect, the method of the present invention includes the treatment of ovarian cancer. In another aspect, the method of the present invention includes the treatment of gastric or pancreatic cancer. In another aspect, the method of the present invention includes the treatment of prostate cancer. In another aspect, the method of the present invention includes the treatment of melanoma. In another aspect, the method of the present invention includes the treatment of small cell and non-small cell lung cancer. In another aspect, the method of the present invention includes the treatment of breast cancer. In another aspect, the method of the present invention includes the treatment of sarcomas, including fibrosarcoma, malignant fibrous histiocytoma, embryonal rhabdomysocarcoman, leiomyosarcoma, neuro-fibrosarcoma, osteosarcoma, synovial sarcoma, liposarcoma, and alveolar soft part sarcoma. In another aspect, the method of the present invention includes the treatment of neoplasms of the central nervous systems, particularly brain cancer. In another aspect, the method of the present invention includes the treatment of lymphomas which include non-Hodgkin's lymphoma, Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma. In another aspect, the method of the present invention includes the treatment of leukemias which include acute lymphocytic leukemia (ALL), acute myeloid leukemia (AML), and myelodysplastic syndromes (MDS). In another aspect, the method of the present invention includes the treatment of multiple myeloma. In other aspects, the method of this invention is used in the prevention or treatment of autoinflammatory diseases driven by IFNs.

The following non-limiting examples are provided to further illustrate the present invention.

### Example 1: Materials and Methods

Mice. Zbp1^{-/-} (Ishii, et al. (2008) Nature 451:725-9), Zbp1^{ΔZα2} (Kesavardhana, et al. (2020) J. Biol. Chem. 295:8325-30), Ripk3^{-/-} (Newton, et al. (2004) Mol. Cell Biol. 24:1464-9), Ripk3^{-/-}Casp8^{-/-} (Oberst, et al. (2011) Nature 471:363-7), Adar1^{fl/fl} (Hartner, et al. (2004) J. Biol. Chem. 279:4894-4902), Nlrp3^{-/-} (Kanneganti, et al. (2006) Nature 440:233-6), Casp11^{-/-} (Kayagaki, et al. (2011) Nature 479:117-21), Asc^{-/-} (Ozoren, et al. (2006) J. Immunol. 176:4337-4342), Tlr3^{-/-} (Alexopoulou, et al. (2001) Nature 413:732-8), Mavs^{-/-} (Kumar, et al. (2006) J. Exp. Med. 203:1795-1803; Suthar, et al. (2010) PLoS Pathog. 6:e1000757), Mda5^{-/-} (Gitlin, et al. (2006) Proc. Natl. Acad. Sci. USA 103:8459-64), Nlrp6^{-/-} (Chen, et al. (2011) J. Immunol. 186:7187-94), Aim2^{-/-} (Jones, et al. (2010) Proc. Natl. Acad. Sci. USA 107:9771-6), and Nlrc4^{-/-} (Mariathasan, et al. (2004) Nature 430:213-8) mice have been previously described. Adar1^{fl/fl}LysM^{cre}Zbp1^{-/-} mice were bred by crossing Adar1^{fl/fl}LysM^{pre} and Zbp1^{-/-} mice. All mice were generated on or extensively backcrossed to the C57/BL6 background. All mice were bred at the Animal Resources Center at St. Jude Children's Research Hospital and maintained under specific pathogen-free conditions. Both male and female mice were used in this study; age- and sex-matched 6- to 9-week-old mice were used for in vitro and 7- to 8-week-old mice were used for in vivo studies. Mice were maintained with a 12-hour light/dark cycle and were fed standard chow. Non-infectious animal studies were conducted under protocols approved by the St. Jude Children's Research Hospital committee on the Use and Care of Animals.

Cell Culture. Primary mouse bone marrow-derived macrophages (BMDMs) were generated from the bone marrow of wild-type and the indicated mutant mice. Cells were grown for 5-6 days in Iscove's Modified Dulbecco's Media (IMDM, Thermo Fisher Scientific) supplemented with 1% non-essential amino acids (Thermo Fisher Scientific), 10% fetal bovine serum (FBS, Biowest), 30% L929-conditioned media, and 1% penicillin and streptomycin (Thermo Fisher Scientific). BMDMs were then seeded into antibiotic-free media at a concentration of 1×10⁶ cells into 12-well plates and incubated overnight.

Cell Stimulation. BMDMs were treated with 5 μM of KPT-330 (Selleckchem) or 5 ng/ml of leptomycin B (Sigma) in the presence and absence of 50 ng/mL of IFN-γ (Peprotech) or 50 ng/mL of IFN-β (PBL Assay) for 12 or 24 hours, as indicated.

Real-time Imaging for Cell Death. The kinetics of cell death were determined using the INCUCYTE® S3 (Essen BioScience) live-cell analysis system. BMDMs (5×10⁵ cells/well) were seeded in 24-well tissue culture plates. Cells were treated with the indicated stimuli and stained with propidium iodide (PI; Life Technologies) following the manufacturer's protocol. The plate was scanned, and fluorescent and phase-contrast images (4 image fields/well) were acquired in real-time every 1 hour from 0 to 24 hours post-treatment. PI-positive dead cells are marked with a red mask for visualization. The image analysis, masking, and quantification of dead cells were done using the software package supplied with the INCUCYTE® imager.

Immunoblot Analysis. Cell lysates and culture supernatants were combined in caspase lysis buffer (containing 1× protease inhibitors (Roche), 1× phosphatase inhibitors (Roche), 10% NP-40, and 25 mM DTT) and 4× sample loading buffer (containing SDS and 2-mercaptoethanol) for immunoblot analysis of caspases. For immunoblot analysis of signaling components, supernatants were removed, and cells were washed once with Dulbecco's phosphate-buffered saline (DPBS, Thermo Fisher Scientific), followed by lysis in radioimmunoprecipitation assay (RIPA) buffer and sample loading buffer. Proteins were separated by electrophoresis through 8-12% polyacrylamide gels. Following electrophoretic transfer of proteins onto polyvinylidene fluoride (PVDF) membranes (Millipore), nonspecific binding was blocked by incubation with 5% skim milk, then membranes were incubated with primary antibodies against: caspase-3 (Cell Signaling Technology (CST), 1:1000), cleaved caspase-3 (CST, 1:1000), caspase-7 (CST, 1:1000), cleaved caspase-7 (CST, 1:1000), caspase-8 (AdipoGen, 1:1000), cleaved caspase-8 CST, 1:1000), caspase-11 (Novus Biologicals, 1:1000), caspase-1 (AdipoGen, 1:1000), GAPDH (CST, 1:1000), ZBP1 (AdipoGen, 1:1000), pMLKL (CST, 1:1000), tMLKL (Abgent, 1:1000), ADAR1 (Santa Cruz Biotechnology, 1:500), tRIPK1 (CST, 1:1000), GSDMD (Abcam, 1:1000), GSDME (Abcam, 1:1000), RIPK3 (CST, 1:1000), pSTAT1 (CST, 1:1000), tSTAT (CST, 1:1000), pIRF3 (CST, 1:1000), HA (Millipore, 1:1000), β-actin (Proteintech, 1:1000). Membranes were then washed and incubated with the appropriate horseradish peroxidase (HRP)-conjugated secondary antibodies (Jackson ImmunoResearch Laboratories; anti-rabbit, 1:5000; anti-mouse 1:5000; and anti-rat, 1:5000). Proteins were visualized using Immobilon Forte Western HRP Substrate (Millipore).

Dot Blot Analysis. For dsRNA analysis, total RNA was isolated from the cells. Equal volumes (2 μl containing 1 μg of RNA) of the RNA were dotted on HYBONDO N+ membrane (GE Healthcare), dried and auto crosslinked in a UV Stratalinker 2400 (Stratagene) two times. The membrane was then blocked in 5% milk in TBST for 1 hour and probed with J2 antibody (1:1000, Scicons) at 4° C. overnight. On the next day, the membrane was washed with TBST 3 times and probed with secondary goat-anti-mouse HRP antibody in 5% milk at room temperature for 1 hour. The total dsRNA was detected using methylene blue.

Immunofluorescence Staining. BMDMs were washed with phosphate-buffered saline (PBS) and fixed in 4% paraformaldehyde for 15 minutes at room temperature, followed by permeabilization for 10 minutes in 0.5% TRITON® X-100 (2-[4-(2,4,4-trimethylpentan-2-yl)phenoxy]ethanol). Cells were blocked in 5% normal goat serum (Life Technologies) for 1 hour at room temperature. Samples were incubated with anti-ZBP1 antibody (1:250 dilution, Adipogen) or anti-J2 antibody (1:100, Millipore) overnight at 4° C. Cells were then washed three times with PBS and incubated with ALEXA FLUOR® 647 fluorescent dye-conjugated antibody against mouse immunoglobulin G (1:250, Invitrogen) and counterstained with DAPI (4′,6-diamidino-2-phenylindol, Life Technologies) for 1 hour at room temperature. Cells were washed three times with PBS and imaged using a Leica SP8 confocal microscope.

Co-Immunoprecipitation Assay. BMDMs (10×10⁶ cells) were seeded into 10-cm dishes and treated with IFN-β or KPT-330 or combination of IFN-β and KPT-330 for 12 hours. Then, the cells were lysed in a buffer containing 20 mM Tris-HCl (pH 7.4), 100 mM NaCl, 30 mM KCl, and 0.1% NP-40. After centrifugation at 16,000 g for 10 minutes, the lysates were incubated with anti-ZBP1 antibody (AdipoGen) with protein A/G PLUS-Agarose (Santa Cruz Biotechnology) for overnight incubation at 4° C. After washing with the above buffer, the immunoprecipitated proteins were harvested by boiling in 1×SDS loading buffer at 100° C. for 5 minutes.

For the overexpression system, plasmids expressing HA-tagged ZBP1-WT, ΔZα, ΔRHIM1, or A311-327 were transfected into 293T cells. After overnight incubation, cells were stimulated with IFN-β for 24 hours. Then cells were lysed in NP-40 lysis buffer (0.1% NP-40, 150 mM NaCl, 50 mM HEPES), and 20 minutes later cell lysates were centrifuged at 13000 rpm for 10 minutes. Supernatant was collected and incubated with 1.5 μg of the indicated primary antibody on a rocking platform at 4° C. After overnight incubation, protein A/G PLUS-Agarose beads were added and incubated for 2 hours. Then the beads were collected by centrifugation after washing with lysis buffer 4 times. Finally, samples were harvested after boiling in 2×SDS loading buffer at 100° C. for 5 minutes.

Microarray Data Analysis. Transcripts were profiled for BMDMs obtained from Adar1^{fl/+}LysM^{cre} and Adar1^{fl/+}LysM^{cre} mice. Total RNA (100 ng) was converted into biotin-labeled cDNA by using an Affymetrix Whole Transcript Plus Expression kit (Thermo Fisher Scientific) and was hybridized to an Affymetrix Clariom S Mouse Genechip Array (Thermo Fisher Scientific). After chips were stained and washed, array signals were normalized and transformed into log 2 transcript expression values by using the robust multi-array average algorithm (Affymetrix Expression Console v1.1). Differential expression was defined by application of a threshold of FDR <0.1 using the Cyber-T t-test. Lists of differentially expressed transcripts were analyzed for ‘functional enrichment’ by using the DAVID bioinformatics database and Ingenuity Pathways Analysis software (QIAGEN). Pathways with altered activity levels were identified by using the Gene Set Enrichment Analysis (GSEA) with curated pathways obtained from The Broad Institute.

Individual expression profiles (log₂ signal) for ADAR1 and ZBP1 were extracted from NCI-60 database. Publicly available transcriptomics data from fetal liver of wild-type and Adar1E861A (editing deficient) mutant E12.5 embryos were obtained from Gene Expression Omnibus database (GEO accession #: GSE58917) (Barrett, et al. (2013) Nucl. Acids Res. 41:D991-5). Heatmap was generated from the 20 most upregulated IFN-stimulated genes (ISGs).

RT-PCR Analysis. Total RNA was extracted using TRIZOL® (Thermo Fisher Scientific) and converted into cDNA by using the High-Capacity cDNA Reverse Transcription Kit (Applied Biosystems). Real-time quantitative PCR was performed on an Applied Biosystems 7500 real-time PCR instrument with 2×SYBR™ Green (Applied Biosystems).

For ERE expression analysis, 5 μg of total RNA extracted from the cells was dissolved in 46 μl of water and mixed with 3.5 μl NaCl (5 M stock). Then, 0.5 μl RNase A (10 mg/ml stock, Thermo Fisher Scientific) or water (as mock) was added to a total volume of 50 μl and mixed, followed by incubation at room temperature for 10 minutes. Then 1 ml acid-guanidinium-phenol based reagent sold under the tradename TRIZOL® was directly added to the mixture to terminate digestion, and the RNA was extracted. The RNA transcripts of selected endogenous retroelements (EREs) were measured by RT-PCR with GAPDH as an internal control. The ratios of (ERE/GAPDH)_{RNAase-A}/(ERE/GAPDH)_{mock} were calculated as fold enrichment. The sequences for qRT-PCR primers are listed in Table 2.

TABLE 2

| | | | SEQ ID |
|---|---|---|---|
| Gene | Primer | Sequence (5′->3′) | NO: |
| mIfi44 | Forward | GAATGGACTTCTCATCTGA | 1 |
| | Reverse | GAACTAAGCTCATCTTGTCT | 2 |
| mIrf7 | Forward | GAGACTGGCTATTGGGGGAG | 3 |
| | Reverse | GACCGAAATGCTTCCAGGG | 4 |
| mIfit3 | Forward | CCTACATAAAGCACCTAGATGGC | 5 |
| | Reverse | ATGTGATAGTAGATCCAGGCGT | 6 |
| mLigp.1 | Forward | CAGGACATCCGCCTTAACTGT | 7 |
| | Reverse | AGGAAGTAAGTACCCATTAGCCA | 8 |
| mIfit1 | Forward | CTGAGATGTCACTTCACATGGAA | 9 |
| | Reverse | GTGCATCCCCAATGGGTTCT | 10 |
| mIfit2 | Forward | AGTACAACGAGTAAGGAGTCACT | 11 |
| | Reverse | AGGCCAGTATGTTGCACATGG | 12 |
| mZbp1 | Forward | AAGAGTCCCCTGCGATTATTTG | 13 |
| | Reverse | TCTGGATGGCGTTTGAATTGG | 14 |
| mGvin1 | Forward | CAGACCACCGAGATGGACTAC | 15 |
| | Reverse | CAAAGACTCTCTTGTTTGCTGC | 16 |
| mIgtp | Forward | CTCATCAGCCCGTGGTCTAAA | 17 |
| | Reverse | CACCGCCTTACCAATATCTTCAA | 18 |
| mIfi203 | Forward | AAGTCAGGCGTCTACAAGTGG | 19 |
| | Reverse | CCTCCCTTTCTTGAGGGTCTT | 20 |
| mMda5 | Forward | AGATCAACACCTGTGGTAACACC | 21 |
| | Reverse | CTCTAGGGCCTCCACGAACA | 22 |
| mIfi2712a | Forward | GCTTGTTGGGAACCCTGTTTG | 23 |
| | Reverse | GGATGGCATTTGTTGATGTGGAG | 24 |
| mHprt | Forward | CTCATGGACTGATTATGGACAGGAC | 25 |
| | Reverse | GCAGGTCAGCAAAGAACTTATAGCC | 26 |
| mMusD | Forward | GATTGGTGGAAGTTTAGCTAGCAT | 27 |
| | Reverse | TAGCATTCTCATAAGCCAATTGCAT | 28 |
| mLine1 | Forward | TTTGGGACACAATGAAAGCA | 29 |
| | Reverse | CTGCCGTCTACTCCTCTTGG | 30 |
| mIAP | Forward | CTTGCCCTTAAAGGTCTAAAAGCA | 31 |
| | Reverse | GCGGTATAAGGTACAATTAAAAGAT | 32 |
| | | ATGG | |
| MuERV-L | Forward | TTTCTCAAGGCCCACCAATAGT | 33 |
| | Reverse | GACACCTTTTTTAACTATGCGAGCT | 34 |

AOM/DSS Model of Colorectal Tumorigenesls. Both male and female mice (littermate controls) were injected with 10 mug ACM (Millipore Sigma) per kg body weight according to previously established protocols (Karki, et al. (2016) Nature 540:583-7). Five days later, 2.5% dextran sulfate sodium (DSS, Affymnetrix) was given in the drinking water for 6 days, followed by regular drinking water for 2 weeks. This cycle was repeated twice with 2% DSS, and mice were sacrificed on day 80. No randomization or blinding was performed.

Histology and Microscopy Analysis. Colons were rolled into a “Swiss roll” and fixed in 10% formalin and then processed and embedded in paraffin by standard techniques. Longitudinal sections of 5 μm thickness were stained with hematoxylin and eosin (H&E) and examined by a pathologist blinded to the experimental groups. Histological scores were assigned based on inflammation, ulceration, hyperplasia, and the extent or severity of the damage. Severity scores for inflammation were assigned as follows: 0, normal (within normal limits); 2, minimal (mixed inflammation, small, focal, or widely separated, limited to lamina propria); 15, mild (multifocal mixed inflammation, often extending into submucosa); 40, moderate (large multifocal lesions within mixed inflammation involving mucosa and submucosa); 80, marked (extensive mixed inflammation with edema and erosions); and 100, severe (diffuse inflammation with transmural lesions and multiple ulcers). Scores for ulceration were assigned as follows: 0, normal (none); 2, minimal (only 1 small focus of ulceration involving fewer than 5 crypts); 15, mild (a few small ulcers, up to 5 crypts); 40, moderate (multifocal ulcers, up to 10 crypts); 80, marked (multifocal to coalescing ulcers involving more than 10 crypts each); and 100, severe (extensive to diffuse, with multiple ulcers covering more than 20 crypts each). Scores for hyperplasia were assigned as follows: 0, normal; 2, minimal (some areas with crypts elongated and increased mitoses); 15, mild (multifocal areas with crypts elongated up to twice the normal thickness, normal goblet cells present); 40, moderate (extensive areas with crypts up to 2 times normal thickness, reduced goblet cells); 80, marked (mucosa over twice the normal thickness, hyperchromatic epithelium, reduced or rare goblet cells, possibly foci of arborization); and 100, severe (mucosa twice the normal thickness, marked hyperchromasia, crowding/stacking, absence of goblet cells, high mitotic index, and arborization). Damage extent scores were assigned as follows: 0, normal (rare or inconspicuous lesions); 2, minimal (less than 5% involvement); 15, mild (multifocal but conspicuous lesions, 5%-10% involvement); 40, moderate (multifocal, prominent lesions, 10%-50% involvement); 80, marked (coalescing to extensive lesions or areas of inflammation with some loss of structure, 50%-90% involvement); and 100, severe (diffuse lesion with effacement of normal structure, >90% involvement).

Melanoma Model. The mouse melanoma cell line B16-F10 (ATCCR CRL-6322™) was cultured in a humidified, 5% CO₂ incubator at 37° C., and grown in Dulbecco's Modified Eagle Medium (DMEM) with 10% FBS and 100 U/mL penicillin/streptomycin. Male and female 6-12-week-old mice were shaved on their lower back and engrafted with B16-F10 melanoma cells by subcutaneously injecting 1×10⁶ cells in 200 μL PBS. Tumors were measured with digital calipers, and tumor volume was calculated using the formula: volume=(length×width²)×½. For treatment with IFN-γ (Peprotech), each mouse was injected interperitoneally with 10 μg of IFN-γ in 100 μl of saline. For treatment with IFN-α or IFN-β, each mouse was injected interperitoneally with 1 μg of IFN-α or IFN-β. For treatment with KPT-330 (Selleckchem), each mouse was orally administered 300 μl of KPT-330 (15 mg/kg) suspended in 0.6% polyoxyalkylene ether sold under the tradename PLURONIC® (Gibco) and 0.6% polyvinylpyrrolidone. The treatment was given on days 8, 10 and 13 after tumor implantation.

Quantification and Statistical Analysis. GraphPad Prism 8.0 software was used for data analysis. Data are shown as mean±SEM. Statistical significance was determined by t tests (two-tailed) for two groups or one-way ANOVA or two-way ANOVA for three or more groups. Survival curves were compared using the log-rank (Mantel-Cox) test. P<0.05 was considered statistically significant.

### Example 2: Interferon Signaling Potentiates the Cell Death Induced by Nuclear Transport Inhibitors

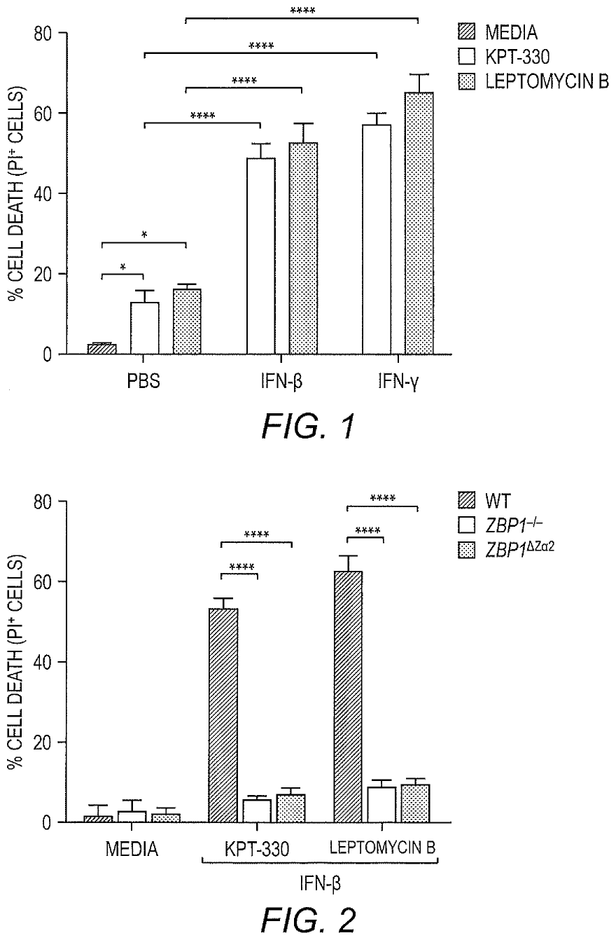

The nucleo-cytoplasmic transport of proteins and RNAs plays a crucial role in maintaining normal cellular functions and homeostasis (Eckmann, et al. (2001) Mol. Biol. Cell 12:1911-24). One of the major eukaryotic nuclear exporters that mediates the transport of proteins with an NES is XPO1. Selective targeting of XPO1-mediated nuclear export by nuclear export inhibitors (NEIs) has shown anti-tumor efficacy (Chari, et al. (2019) N. Engl. J. Med. 381:727-38; Theodoropoulos, et al. (2020) Target Oncol. 15:697-708), which may be due to ability of NETs to induce cancer cell death (Mutka, et al. (2009) Cancer Res. 69:510-517). Therefore, molecularly defining the cell death pathway induced by NEIs, which has previously been thought to be immunologically silent apoptosis (Parikh, et al. (2014) J. Hematol. Oncol. 7:78), is critical to understand the molecular mechanisms by which NEIs inhibit tumorigenesis. To understand the ability of the NEIs to induce cell death, bone marrow-derived macrophages (BMDMs) were treated with the NEIs KPT-330 or leptomycin B (LMB). Treatment with either KPT-330 or LMB for 24 hours induced a low level of cell death in BMDMs at the concentration used (FIG. 1). To investigate whether NEIs can induce inflammatory cell death in the form of pyroptosis, the cleavage of gasdermin D (GSDMD) was monitored. Treatment with NEIs led to production of a small amount of the active P30 fragment of GSDMD that can form membrane pores to induce pyroptosis. GSDMD can be processed to release this P30 fragment by caspase-1, downstream of inflammasome activation, or by caspase-11 (He, et al. (2015) Cell Res. 25:1285-98; Kayagaki, et al. (2015) Nature 526:666-76; Shi, et al. (2015) Nature 526:660-65). Consistent with the amount of GSDMD P30 produced, there was minimal cleavage of caspase-1 and caspase-11. Another member of the gasdermin family, GSDME, has also been shown to induce pyroptosis under specific conditions (Wang, et al. (2017) Nature 547:99-103). It was observed that BMDMs treated with KPT-330 or LMB also displayed cleavage of GSDME, demonstrating that the NEIs induced pyroptosis in BMDMs.

In addition to pyroptosis, it was also found that KPT-330 or LMB induced apoptosis in BMDMs, as evidenced by the cleavage of apoptotic caspases caspase-8, -3, and -7. Furthermore, recent studies have shown that activation of caspase-3 and -7 can inactivate GSDMD by processing it to produce a P20 fragment (Chen, et al. (2019) J. Immunol. 186:7187-94; Taabazuing, et al. (2017) Cell Chem. Biol. 24:507-514 e504), which was also observed in these analyses.

It was subsequently determined whether the NEIs induced necroptosis. Cells stimulated with KPT-330 or LMB showed a low level of phosphorylation of MLKL, suggesting that necroptosis is occurring. Cleavage of total RIPK1, which is involved in regulating apoptosis and necroptosis (Newton, et al. (2019) Nature 574:428-31), was also noted.

Several studies have shown that IFN signaling potentiates pyroptosis, apoptosis, necroptosis, and PANoptosis in a context-dependent manner (Gurung, et al. (2012) J. Biol. Chem. 287:34474-83; Karki, et al. (2018) Cell 173:920-933 e913; Karki, et al. (2020) J. Immunol. 204:2514-22; Karki, et al. (2020) JCI Insight 5(12):e136720; Karki, et al. (2021) Cell 184:149-168 e117; Malireddi & Kanneganti (2013) Front Cell Infect. Microbiol. 3:77; Man, et al. (2015) Nat. Immunol. 16:467-75; Man, et al. (2016) Cell 167:382-96 e317; Sarhan, et al. (2019) Cell Death Differ. 26:332-347; Yang, et al. (2020) Cell Mol. Immunol. 17:356-68). IFN-based therapies have been used historically to treat cancer in both preclinical and clinical studies (Arico, et al. (2019) Cancers (Basel) 11(12):1943). Additionally, IFN signaling-mediated PANoptosis suppresses the development of colorectal cancer (Karki, et al. (2020) JCI Insight 5(12):e136720). Treatment with IFN-β or IFN-γ in combination with KPT-330 or LMB increased the incidence of cell death compared to treatment with KPT-330 or LMB alone (FIG. 1), indicating that IFN signaling potentiates the cell death induced by NEIs. In line with the incidence of cell death, treatment with IFN-3 or IFN-γ potentiated KPT-330- or LMB-induced cleavage of caspase-1 and GSDME (pyroptosis), cleavage of caspase-8, -3, and -7 (apoptosis), and phosphorylation of MLKL (necroptosis). Collectively, these data indicate that combining IFNs with NEIs sensitizes the cells to undergo inflammasome activation and PANoptosis, a form of inflammatory cell death involving the components of pyroptosis, apoptosis, and necroptosis.

### Example 3: ZBP1 Engages RIPK3 Signaling to Activate the NLRP3 Inflammasome and PANoptosis Induced by NEIs

Innate immune sensors play critical roles in activating the inflammasome and driving cell death (Man, et al. (2017) Immunol. Rev. 277:61-75). Since combining IFNs and NEIs induced caspase-1 cleavage, it was first determined whether the cytosolic sensors that are known to assemble inflammasomes activate caspase-1 and induce cell death. The caspase-1 cleavage induced by IFNs and NEIs was impaired in Nlrp3^{-/-} and Asc^{-/-} BMDMs, but not in Nlrc4^{-/-}, Aim2^{-/-} or Casp1^{-/-} BMDMs, indicating that the combination of IFNs with NEIs activates the NLRP3 inflammasome. Despite the defective caspase-1 cleavage, cell death was not impaired in Nlrp3^{-/-} BMDMs, suggesting that other molecules are involved in regulating the cell death. Next, other innate immune sensors that are known to regulate inflammasome activation and cell death in various contexts (Man, et al. (2017) Immunol. Rev. 277:61-75) were screened. It was found that BMDMs lacking ZBP1 had reduced cell death compared with wild-type BMDMs after IFN-β and KPT-330 treatment (FIG. 2). Consistent with this protection, Zbp1^{-/-} BMDMs showed reduced cleavage of caspase-1 and impaired activation of the pyroptotic molecule GSDME; apoptotic caspases (caspase-8, -3, and -7); and the necroptotic molecule MLKL, suggesting that ZBP1 is required for NEI-induced activation of the NLRP3 inflammasome and PANoptosis.

Sensing of viral or endogenous Z-RNA by the Zα domain of ZBP1 triggers NLRP3 inflammasome activation, PANoptosis, and perinatal lethality in mice, indicating that the Zα domain is crucial to regulate the immune responses driven by ZBP1 (Devos, et al. (2020) J. Exp. Med. 217(7):e20191913; Jiao, et al. (2020) Nature 580:391-5; Kesavardhana, et al. (2020) J. Biol. Chem. 295:8325-30). Therefore, the role of the Zα domain of ZBP1 in driving the inflammasome activation and PANoptosis induced by NEIs was investigated. Similar to Zbp1^{-/-} BMDMs, cells lacking the Zα domain of ZBP1 showed reduced cell death compared with that of wild-type BMDMs after treatment with IFN-β and KPT-330 or IFN-β and LMB (FIG. 2). Consistent with the reduced cell death, Zbp1^{ΔZα2} BMDMs showed impaired cleavage of caspase-1 and impaired activation of the pyroptotic molecule GSDME; apoptotic caspases (caspase-8, -3, and -7); and the necroptotic molecule MLKL, suggesting that the Zα domain of ZBP1 is required for NEI-induced activation of the NLRP3 inflammasome and PANoptosis.

ZBP1 activation leads to its interaction with RIPK3 and recruitment of caspase-8 and caspase-6 to form a cell death signaling scaffold that drives NLRP3 inflammasome activation and PANoptosis (Kesavardhana, et al. (2020) J. Biol. Chem. 295:8325-30; Kuriakose, et al. (2016) Sci. Immunol. 1(2):aag2045; Rebsamen, et al. (2009) EMBO Rep. 10:916-22; Zheng, et al. (2020) Cell 181:674-87 e613). It was therefore assessed whether RIPK3 and caspase-8 had any role in the NEI-induced cell death pathways. Similar to Zbp1^{-/-} or Zbp1^{ΔZα2} BMDMs, cells lacking RIPK3 showed reduced cell death and activation of PANoptotic molecules compared with that of wild-type BMDMs after IFN-β and KPT-330 or IFN-β and LMB treatment. Significant differences in the extent of cell death were not observed between Ripk3^{-/-} or Ripk3^{-/-}Casp8^{-/-} BMDMs, indicating that the phenotype observed in Ripk3^{-/-}Casp8^{-/-} BMDMs is largely attributed to the RIPK3 deficiency.

Altogether, these data indicate that the Zα domain of ZBP1 is required to activate RIPK3 signaling to induce activation of the NLRP3 inflammasome and PANoptosis in response to the combination of IFNs and NEIs.

### Example 4: ADAR1 Suppresses ZBP1-Mediated NLRP3 Inflammasome Activation and PANoptosis

In addition to ZBP1, the only other mammalian protein that contains a Zα domain is the ADAR1-p150 splice isoform (Schwartz, et al. (1999) Science 284:1841-5). However, the relationship between these two molecules is not known. Given that both ADAR1-p150 and ZBP1 contain Zα domains, it is tempting to speculate that ADAR1 can act similarly to ZBP1 and promote inflammasome activation and inflammatory cell death, PANoptosis. In addition to its Zn domain, ADAR1-p150 also contains a nuclear export signal (NES); inhibition of the NES by XPO1-specific NEIs such as KPT-330 or LMB induces nuclear accumulation of ADAR1 (Poulsen, et al. (2001) Mol. Cell Biol. 21:7862-71).

Since ADAR1-deficient mice are embryonically lethal (Hartner, et al. (2004) J. Biol. Chem. 279:4894-4092; Hartner, et al. (2009) Nat. Immunol. 10:109-115; Liddicoat, et al. (2015) Science 349:1115-1120; Wang, et al. (2000) Science 290:1765-68), BMDMs were derived from mice lacking ADAR1 in myeloid cells (Adar1^{fl/fl}LysM^{cre}, referred to as Adar1^{-/-} BMDMs herein) to investigate the role of ADAR1 in NEI-mediated inflammasome activation and inflammatory cell death. Increased cell death induced by the combination of IFN-3 and KPT-330 or IFN-β and LMB was observed in the cells lacking ADAR1 compared with those of wild-type and Zbp1^{-/-} cells. Moreover, analysis of real-time cell death revealed accelerated dynamics of cell death in ADAR1-deficient BMDMs compared with those of wild-type and Zbp1^{-/-} BMDMs.

Despite the fact that both ADAR1-p150 and ZBP1 are IFN-inducible and contain Zα domains (Kuriakose, et al. (2018) J. Immunol. 200:1489-95; Patterson & Samuel (1995) Mol. Cell Biol. 15:5376-88; Schwartz, et al. (1999) Science 284:1841-5), they showed contrasting phenotypes in terms of cell death. To understand how ADAR1 deficiency contributes to enhanced cell death, a microarray analysis was performed to identify differentially regulated ISGs in wild-type and ADAR1-deficient BMDMs under basal condition. Consistent with the previous findings (Barrett, et al. (2013) Nucl. Acids Res. 41:D991-5; Liddicoat, et al. (2015) Science 349:1115-20), ADAR1-deficient macrophages showed increased expression of ISGs including Ifi4.4, Irf7, Ifit3, Ligp1, Ifit2, Ifit2, Zbp1, Gvin1, Igtp, Ifi203, Mda5 and Ifi2712a. Indeed, Zbp1 was one of the most highly upregulated ISGs in ADAR1-deficient BMDMs. The increased expression of ISGs in Adar1^{-/-} BMDMs was further validated using RT-PCR. Increased expression of ZBP1 in ADAR1-deficient BMDMs was further confirmed by western blot analysis and immunofluorescence. Although treatment with IFN-β induced ZBP1 expression in wild-type BMDMs, notable upregulation of ZBP1 in Adar1^{-/-} BMDMs was not observed. Moreover, the increased phosphorylation and expression of IRF3 and STAT1 observed in Adar1^{-/-} BMDMs suggests that the upregulated expression of ISGs is due to increased IFN signaling. Furthermore, analysis of a publicly available dataset revealed increased ZBP1 expression together with other ISGs in ADAR1-deficient fibroblasts. All these findings suggest that deficiency of ADAR1 leads to increased ZBP1 expression.

Since ADAR1-deficient BMDMs show high expression of ZBP1 basally, these cells could be prone to NEI-induced cell death even without IFN treatment. As expected Adar1^{-/-} BMDMs showed increased cell death upon treatment with KPT-330 or LMB alone. Notably, Adar1^{-/-} BMDMs had increased cleavage of caspase-1 and activation of the pyroptotic molecule GSDME; apoptotic caspases (caspase-8, -3, and -7); and the necroptotic molecule MLKL after treatment with KPT-330 or LMB alone, suggesting that ADAR1 suppresses NLRP3 inflammasome activation and PANoptosis induced by NEIs.

Subsequently, cells lacking both ADAR1 and ZBP1 were generated to further confirm that increased ZBP1 expression contributes to the accelerated inflammasome activation and PANoptosis in the absence of ADAR1. BMDMs deficient in both ADAR1 and ZBP1 showed similar cell death to that of wild-type cells in response to KPT-330 or LMB. Furthermore, NLRP3 inflammasome activation and PANoptosis were inhibited upon deletion of ZBP1 in Adar1^{-/-} BMDMs treated with NEIs. Overall, these results show that loss of ADAR1 leads to increased expression of ZBP1, which triggers accelerated inflammasome activation and cell death induced by NEIs.

### Example 5: IFN and NEI Treatment Induces Endogenous dsRNA Expression to Activate ZBP1

Despite having upregulated ZBP1 expression basally, Adar1^{-/-} BMDMs still need KPT-330 or LMB treatment to drive inflammatory cell death. It was therefore hypothesized that KPT-330 or LMB would induce the release of triggers that activate ZBP1. Previous studies suggested that ZBP1 senses genomic RNA from influenza A virus (IAV) through its Zα domains to trigger cell death. Considering there was no viral infection in Adar1^{-/-} BMDMs, whether de-repressed endogenous dsRNA could function as a trigger to prime ZBP1 and induce cell death was investigated. An increase in the amount of dsRNA present in Adar1^{-/-} BMDMs was observed upon treatment with KPT-330 or LMB by using the J2 antibody for dot-blot analysis and immunofluorescence. Furthermore, dsRNAs from endogenous retroelements (EREs) have been shown to bind ZBP1 to induce inflammatory cell death (Wang, et al. (2020) Nature 580:386-90). To test whether ERE-derived dsRNA could be induced by KPT-330 or LMB treatment, expression of MuS-D, Line1, ERV-L and IAP was examined in Adar1^{-/-} cells. Treatment of Adar1^{-/-} BMDMs with KPT-330 or LMB increased the expression of these EREs by approximately 2- to 12-fold. However, wild-type BMDMs require the combination of IFNs with KPT-330 or LMB to induce robust amounts of cell death. Therefore, the levels of dsRNA and EREs in KPT-330- or LMB-treated wild-type BMDMs were examined with or without IFN-β priming. Treatment of wild-type cells with KPT-330 or LMB alone slightly increased the amounts of dsRNA compared with media-treated wild-type cells. However, the combination of IFN-β and KPT-330 or IFN-β and LMB robustly increased dsRNA compared with media-, KPT-, or LMB-treated wild-type cells. Moreover, the expression of EREs such as Mus-D, Line1 and ERV-L was slightly upregulated in cells treated with KPT-330 or LMB alone but robustly increased in cells treated with the combination of IFN-β and KPT-330 or IFN-β and LMB (10- to 30-fold).

### Example 6: ADAR1 Competes with RIPK3 for ZBP1 Binding to Suppress Inflammasome Activation and Cell Death

Sensing of Z-RNA by the Zα domain of ZBP1 is followed by the RHIM domain of ZBP1 interacting with the corresponding RHIM domain of RIPK3 to drive cell death (Devos, et al. (2020) J. Exp. Med. 217(7):e20191913; Jiao, et al. (2020) Nature 580:391-95; Kesavardhana, et al. (2020) J. Biol. Chem. 295:8325-30; Rebsamen, et al. (2009) EMBO Rep. 10:916-22). Since both ZBP1 and ADAR1 contain Zα domains, it is possible that ADAR1 interacts with ZBP1 via the Zα domains, thereby limiting the availability of ZBP1 for binding to RIPK3 and inhibiting the subsequent cell death. It was therefore determined whether ZBP1 could interact with ADAR1 endogenously in wild-type BMDMs under different conditions. It was observed that the expression of ADAR1 and ZBP1 was upregulated in response to IFN-β stimulation. In line with the low expression of ZBP1 and ADAR1 before IFN induction, no interaction was observed between ZBP1 and ADAR1 under basal conditions. However, a robust interaction of ZBP1 with ADAR1 was observed, but not with RIPK3, in wild-type cells stimulated with IFN-β. Although wild-type cells treated with KPT-330 alone showed some interaction of ZBP1 with both ADAR1 and RIPK3, stimulation with the combination of IFN-β and KPT-330 reduced the interaction of ZBP1 with ADAR1 and increased the interaction of ZBP1 with RIPK3. The increased interaction between ZBP1 and RIPK3 in response to IFNs and NEIs together compared with NEIs alone likely explains the increased cell death caused by this combination.

Since the homotypic interaction between ZBP1 and RIPK3 occurs via their RHIM domains (Rebsamen, et al. (2009) EMBO Rep. 10:916-22), it is likely that ZBP1 can interact with ADAR1 via their Zα domains. To determine whether the Zα domain of ZBP1 is critical for the interaction of ZBP1 with ADAR1, HA-tagged mutant ZBP1 constructs lacking Zα (ΔZα), RHIM (ΔRHIM), or C-terminal (A311-327) domains were constructed and overexpressed in 293T cells. Wild-type ZBP1 was co-immunoprecipitated with ADAR1. While mutant ZBP1 lacking the RHIM or C-terminal domains also co-immunoprecipitated with ADAR1, mutant ZBP1 lacking the Zα domains failed to efficiently pull down ADAR1. Similarly, ADAR1 co-immunoprecipitated with wild-type ZBP1 and the mutants lacking RHIM or C-terminal domains, but not the mutant lacking Zα domains, indicating that ZBP1 interacts with ADAR1 via its Zα domain. Although the importance of the interaction between ZBP1 and RIPK3 is well established in activating cell death pathways, the significance of the interaction between ZBP1 and ADAR1 that was identified in this study was not known. Given that ADAR1 suppresses the ZBP1/RIPK3-mediated cell death, ADAR1 could potentially compete with RIPK3 for binding to ZBP1. To determine whether this occurs, the endogenous interaction between ADAR1 and ZBP1 in the presence and absence of RIPK3 was compared, as well as the interaction between RIPK3 and ZBP1 in the presence and absence of ADAR1. The results of this analysis did not detect any interaction between ADAR1 and ZBP1 or between RIPK3 and ZBP1 in wild-type BMDMs under basal conditions. Upon treatment with IFN-β and KPT-330, ZBP1 interacted with both ADAR1 and RIPK3. However, the interaction between ZBP1 and RIPK3 was increased in the absence of ADAR1. Furthermore, the interaction between ZBP1 and ADAR1 was increased in Ripk3^{-/-} cells compared with that of wild-type cells, indicating that ADAR1 competes with RIPK3 for binding to ZBP1. Overall, ADAR1 interacts with ZBP1 via the ZBP1 Zα domain, and this interaction limits the availability of ZBP1 to bind with RIPK3 to activate the inflammatory cell death pathways.

### Example 7: ADAR1 Promotes Tumorigenesis in a ZBP1-Dependent Manner

Dysregulated cell death and inflammatory responses are associated with tumorigenesis. Resistance to cell death, particularly apoptosis, is one of the founding hallmarks of cancer (Green & Evan (2002) Cancer Cell 1:19-30; Hanahan & Weinberg (2000) Cell 100:57-70; Hanahan & Weinberg (2011) Cell 144:646-74) and can explain the failure of many existing anti-cancer therapies (Delbridge, et al. (2012) Cold Spring Harb. Perspect. Biol. 4(11):a008789). Therefore, alternative mechanisms that drive programmed cell death (PCD) such as PANoptosis can be effective in eliminating cancer cells. Indeed, when apoptosis is not effective, alternative PCD pathways pyroptosis or necroptosis and inflammasome activation are beneficial to eliminate cancer cells (Chefetz, et al. (2019) Cell Rep. 26:3061-75 e3066; Fu, et al. (2013) BMC Cancer 13:580; Karki & Kanneganti (2019) Nat. Rev. Cancer 19:197-214; Karki, et al. (2017) Cancer Immunol. Res. 5:94-99; Lage, et al. (2001) FEBS Lett. 494:54-59; Nagarajan, et al. (2019) Transl. Oncol. 12:925-31; Wang, et al. (2017) Nature 547:99-103). However, certain host molecules may inhibit these PCD pathways, promoting the development of cancers. Since ADAR1 inhibited inflammasome activation and PANoptosis, it was hypothesized that ADAR1 can promote tumorigenesis.

To investigate the role of ADAR1 in tumorigenesis, the expression of ADAR1 and ZBP1 was determined in several human cancer lines. All cancer cell lines from the NCI-60 panel showed high expression of ADAR1 compared to that of ZBP1, suggesting that ADAR1 and ZBP1 could differentially modulate tumorigenesis. To further understand the function of ADAR1 in tumor development, the established azoxymethane/dextran sodium sulfate (AOM/DSS) model of colorectal tumorigenesis in mice (Karki, et al. (2016) Nature 540:583-7) was used. Mice were injected with 10 mg AOM per kg body weight on day 1, then given 2.5% dextran sulfate sodium in the drinking water 5 days later for 6 days, followed by regular drinking water for 2 weeks. This DSS dosing cycle was repeated twice with 2% DSS, and mice were sacrificed on day 80. Mice were monitored for body weight change and prevalence of tumors. The mice lacking ADAR1 in myeloid cells lost more body weight compared with littermate controls after each cycle of DSS. Although Adar1^{fl/fl}LysM^{cre} mice lost more body weight, the colons of the mice had a lower tumor burden in terms of both number of tumors and tumor size compared with the littermate control mice. Histopathological analysis revealed reduced thickening of the colons in Adar1^{fl/fl}LysM^{cre} mice compared with colons from the control mice. Histological hallmarks associated with inflammation, ulceration, hyperplasia, and severity of damage were less frequently observed in the middle and distal colons of Adar1^{fl/fl}LysM^{cre} mice compared with the corresponding regions in the control mice. Additionally, Adar1^{fl/fl}LysM^{cre} mice had low grade dysplasia, while many of the control mice had high grade dysplasia. All these data indicate that ADAR1 deficiency in myeloid cells inhibits colorectal tumorigenesis. This analysis of the tumor promoting function of ADAR1 was further extended to melanoma. Adar1^{fl/fl}LysM^{cre} and the littermate control mice were subcutaneously injected with melanoma cells and monitored for 2 weeks for tumor growth. It was found that Adar1^{fl/fl}LysM^{cre} mice exhibited significantly impaired tumor growth and lower tumor weight compared with the control mice. Together, these findings demonstrate that ADAR1 promoted tumorigenesis in the development of colorectal cancer and melanoma.

The role of ZBP1 was then examined in melanoma growth by injecting B16 melanoma cells into wild-type, Zbp1^{-/-} and Zbp1^{ΔZα2} mice. Both Zbp1^{-/-} and Zbp1^{ΔZα2} mice developed larger melanoma tumors compared with wild-type mice, suggesting that the Zα2 domain of ZBP1 contributes to the inhibition of melanoma development. In contrast to ZBP1 deficiency, loss of ADAR1 in myeloid cells inhibited the melanoma development. To determine whether this suppression of tumorigenesis in ADAR1-deficient mice was due to ZBP1, Adar1^{fl/+}, Adar1^{fl/fl}LysM^{cre} and Adar1^{fl/fl}LysM^{cre}Zbp1^{-/-} mice were injected with B16 melanoma cells. Again, mice lacking ADAR1 in myeloid cells showed reduced melanoma growth compared with wild-type mice. However, deficiency of ZBP1 in the mice lacking ADAR1 in myeloid cells failed to inhibit melanoma growth. Deletion of the Zα2 domain of ZBP1 yielded similar results, suggesting that the ZBP1 Zα2 domain is crucial in suppressing melanoma growth in mice lacking ADAR1 in myeloid cells.

The role of ZBP1 in suppressing tumorigenesis in the absence of ADAR1 was further investigated in a model of AOM-DSS-induced colorectal cancer. Consistent with the melanoma model, the loss of ZBP1 or deletion of the Zα2 domain of ZBP1 in mice lacking ADAR1 in myeloid cells resulted in similar tumor growth to that seen in Adar1^{fl/+} mice. These data indicate that ZBP1 is crucial in suppressing tumorigenesis in the absence of ADAR1.

### Example 8: Combined Treatment with IFN and NEI Regresses Tumors

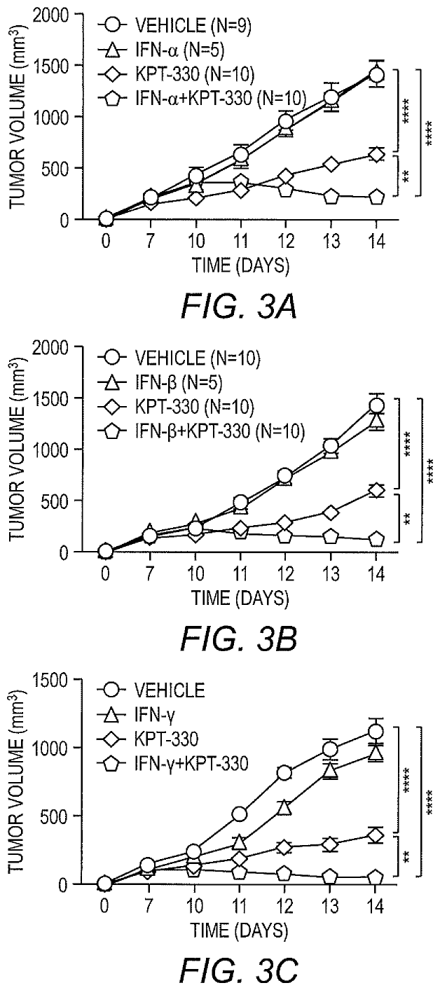

The inflammatory cell death induced by the combination of IFN and NEIs was dependent on ZBP1. Therefore, the efficacy of IFN and NEIs in treating melanoma was evaluated. Wild-type mice treated with KPT-330 8 days after B16 melanoma cell implantation showed reduced melanoma growth. However, mice treated with the combination of IFN-α (FIG. 3A), IFN-β (FIG. 3B), or IFN-γ (FIG. 3C), and KPT-330 had a significantly improved regression of their tumors. To determine whether the tumor regression was dependent on ZBP1, the combination of IFN-γ and KPT-330 was administered to wild-type and Zbp1^{-/-} mice. IFN-γ and KPT-330 failed to regress melanoma in Zbp1^{-/-} mice. Altogether, these data indicate that IFN and KPT-330 combination therapy is a superior treatment combination and that ZBP1 suppresses tumorigenesis in the absence of ADAR1.

What is claimed is:

1. A method for treating cancer comprising administering to a subject in need thereof an effective amount of (i) one or more interferons or (ii) one or more agents that upregulate interferon production; and one or more nuclear export inhibitors thereby treating the subject's cancer.

2. The method of claim 1, wherein the one or more interferons comprise IFN-α, IFN-β, IFN-γ, or combinations thereof.

3. The method of claim 1, wherein the one or more nuclear export inhibitors comprise a leptomycin, a ratjadone, an anguinomycin, callystatin, valtrate, oridonin, acetoxychavicol acetate, curcumin, gonionthalamin, piperlongumine, plumbagin, CBS-9106, KPT-185, KPT-249, KPT-251, KPT-276, KPT-301, KPT-330, KPT-335 or KPT-8602.

4. The method of claim 1, wherein the one or more agents that upregulate interferon production comprise amidobenzimidazoles, flavone acetic acid analogues or cyclic dinucleotides.

5. The method of claim 1, wherein the cancer is a head and neck cancer, liver cancer, intestinal cancer, ovarian cancer, gastric cancer, pancreatic cancer, prostate cancer, melanoma, lung cancer, breast cancer, sarcoma, cancer of the central nervous system, lymphoma, leukemias or myeloma.

6. A composition comprising an effective amount of one or more nuclear export inhibitors and (i) one or more interferons or (ii) one or more agents that upregulate interferon production, the effective amount being synergistic when compared to an effect of the one or more nuclear export inhibitors and the (i) one or more interferons or (ii) one or more agents that upregulate interferon production when used alone.

7. The composition of claim 6, wherein the one or more interferons comprise IFN-α, IFN-β, IFN-γ, or combinations thereof.

8. The composition of claim 6, wherein the one or more nuclear export inhibitors comprise a leptomycin, a ratjadone, an anguinomycin, callystatin, valtrate, oridonin, acetoxychavicol acetate, curcumin, gonionthalamin, piperlongumine, plumbagin, CBS-9106, KPT-185, KPT-249, KPT-251, KPT-276, KPT-301, KPT-330, KPT-335 or KPT-8602.

9. The composition of claim 6, wherein the one or more agents that upregulate interferon production comprise amidobenzimidazoles, flavone acetic acid analogues or cyclic dinucleotides.
